(19) United States
(12) Patent Application Publication
(10) Pub. No.: US 2015/0020447 A1
(43) Pub. Date: Jan. 22, 2015

(54) METHOD AND DEVICE FOR ERADICATING INVASIVE, PROLIFERATING PLANTS

(71) Applicant: GECO INGENIERIE, LAUDUN L'ARDOISE (FR)

(72) Inventor: Frederic Roure, Laudun L'Ardoise (FR)

(21) Appl. No.: 14/384,093

(22) PCT Filed: Mar. 12, 2013
(86) PCT No.: PCT/FR2013/050514

§ 371 Date: Sep. 9, 2014

(30) Foreign Application Priority Data
Mar. 12, 2012 (FR) 12 52174

(51) Int. Cl. A01M 21/04; A61L 2/12

(52) U.S. Cl. CPC A01M 21/04; A61L 2/12
USPC 47/58.1 R; 250/492.1

(57) ABSTRACT

A mobile device for eradicating proliferating terrestrial plants has a feeder to introduce the plants to be treated and a generator to generate electromagnetic waves for sterilizing the plants within the layer of plants. The generator can be configured to emit microwaves and/or to heat the seeds of the plants to a temperature above 60° C. The mobile device can include a hopper to receive the plants, a separator to remove metal and/or stones, and a grinder to grind the plants, which is arranged upstream from the generator.

### TECHNICAL FIELD OF INVENTION

The present invention envisages a method and a device for eradicating invasive, proliferating plants. It applies, in particular, to the cleaning of river banks and/or to suppressing unwanted plants from imported and/or backfilled materials (e.g. topsoil, compost).

### BACKGROUND OF THE INVENTION

Invasive, proliferating exotic plants currently propagate along river banks, especially in Europe. For example, some of these plants are the Primrose Willows (ludwigia sp.) and Japanese Knotweed (Sackalines sp.), Buddleia Davidii, Caulerpas, Impatiens, and Ragweeds. The resulting invasion is harmful to the biodiversity of these areas and damages the habitat for the residents and animals who live there or close to them.

Uprooting these plants mechanically or by hand is a tricky operation. Their proliferation means that grubbing-out operations must be repeated. Above all, the waste obtained during these grubbing-out operations requires transport and treatments that are complex and expensive.

Methods and devices for sterilizing the surface of the soil by using microwaves are known, for example those presented in documents WO 2008/057215, NL 8 900 730 and U.S. 2002/090268. However, these methods only allow a shallow depth of the ground to be treated and are therefore not effective. In addition, these methods and devices are not suitable for the treatment of aquatic plants and are strongly disrupted by the presence of metals at the surface of the soil.

### OBJECT AND SUMMARY OF THE INVENTION

The present invention seeks to remedy all or some of these drawbacks by providing a device and a method that leave this waste, rendered sterile, behind and allow other plants, normally found in Europe, to become established.

To this end, according to a first aspect, the present invention envisages a mobile device for eradicating proliferating and/or invasive terrestrial plants, which comprises:
a means for introducing the plants to be treated; and
a means for generating electromagnetic waves for sterilizing the plants within the layer of plants.

Thanks to these arrangements, invasive, proliferating plants can be sterilized directly where they are collected. In addition, the residues, i.e. the sterilized plants, can be left behind and used to nourish the renaturalized plants planted in their place. It is therefore unnecessary to transport these invasive plants, or to store them out of the ground.

In some embodiments, the means for introducing the plants to be treated comprises a means for setting up a layer of plants to be treated.

It is thus possible to adapt the thickness of the layer to the power of the electromagnetic waves.

In some embodiments, the means for generating electromagnetic waves emits microwaves.

Thanks to these arrangements, the seeds are heated until they are deactivated.

In some embodiments, the means for generating electromagnetic waves emits microwaves with a frequency between 500 MHz and 5 GHz.

The shorter the wavelength of the microwaves, the farther the microwaves penetrate.

In some embodiments, the means for generating electromagnetic waves emits microwaves with a frequency between 500 MHz and 1 GHz.

Using these preferred wavelengths makes it possible to not have to reduce the thickness of the layer of matter to be treated.

In some embodiments, the means for generating electromagnetic waves is designed to heat the seeds of the plants to a temperature above 60° C.

Thanks to these arrangements, the seeds are heated until they are deactivated.

In some embodiments, the eradication device that is the subject of the present invention comprises, in addition, a hopper for receiving the plants, and a means for removing metal and/or stones.

Thanks to these arrangements the metals, which can interact with the electromagnetic waves generated by the generating means, are extracted upstream of this generation means.

In some embodiments, the device that is the subject of the invention comprises, in addition, a means for making the thickness of the layer of plants to be treated uniform, upstream from the means for generating electromagnetic waves.

Thanks to these arrangements, the effectiveness of the electromagnetic waves is increased.

In some embodiments, the device that is the subject of the present invention comprises, in addition, a means for grinding the plants, which is arranged upstream from the means for generating electromagnetic waves.

Thanks to these arrangements, the effectiveness of the electromagnetic waves is increased and the plants are also killed by grinding.

In some embodiments, the device that is the subject of the present invention comprises a means for water-jetting the plants, configured to partially dry the plants before they pass in front of the means for generating electromagnetic waves.

It is noted here that water-jetting consists of separating, at least partially, the water from the plants to be treated. The water-jetting is a technique positioned upstream of the heat treatment method, which allows plants and silts to be pumped to a drainage station. The water-jetting, carried out using a boat, allows the plants and their seeds, the silt and the gravel to be pumped with large volumes of water

According to a second aspect, the present invention envisages a method for eradicating proliferating and/or invasive terrestrial plants, which comprises:
a step of collecting plants;
a step of introducing the plants to be treated; and
a step of generating electromagnetic waves for sterilizing the plants within the layer of plants.

As the features, advantages and aims of the method that is the subject of the present invention are similar to those of the device that is the subject of the present invention, they are not repeated here.

### BRIEF DESCRIPTION OF THE DRAWINGS

Other advantages, aims and characteristics of the present invention will become apparent from the description that will follow, made, as an example that is in no way limiting, with reference to the drawings included in an appendix, wherein:

FIG. 1 represents, schematically, a first particular embodiment of the device for eradicating plants;

FIG. 2 represents, in the form of logical diagrams, steps in a first particular embodiment of the eradication method that is the subject of the present invention;

FIG. 3 represents, in the form of logical diagrams, steps in a second particular embodiment of the method for eradicating plants that is the subject of the present invention;

FIG. 4 represents, in the form of logical diagrams, steps in a third particular embodiment of the method for eradicating plants that is the subject of the present invention;

FIG. 5 represents, schematically, a second particular embodiment of the device for eradicating plants.

### DETAILED DESCRIPTION OF EMBODIMENTS

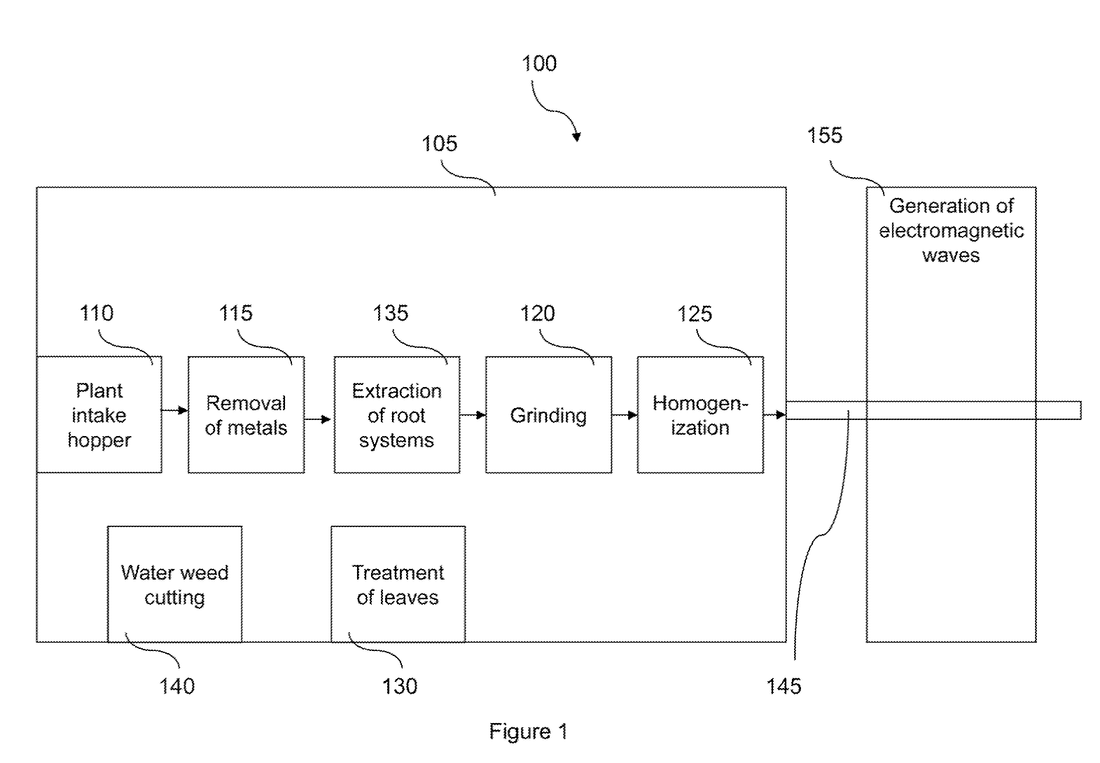

FIG. 1 shows a device 100 for eradicating plants, which comprises:
a means 105 for setting up a mass of plants to be treated; and
a means 155 for generating electromagnetic waves for sterilizing the plants within the layer of plants.

The means 155 for generating electromagnetic waves emits microwaves. The means 155 for generating electromagnetic waves is designed to heat and maintain at temperature the mass of plants and, in particular, the seeds of the plants to a temperature above 60° C., and preferably above 100° C. The seeds are thus heated until they are deactivated and, preferably, all of the plant's tissues are killed.

The means 105 for setting up a mass of plants to be treated comprises, in the case where this mass is formed into a layer:
a hopper 110 for receiving the plants;
a means 115 for removing metal and/or stones (for removing solid waste and scrap, at least one trommel and at least one electromagnet are utilized, for example); a trommel-type circular particle-size separator allows solid waste and large blocks, cleared of any earth or organic fragments, to be removed; this trommel, equipped with different sizes of openings, allows the materials to be sorted according to their block/particle size distribution and their weight;
a means 120 for grinding the plants, which makes the residual plant mass uniform;
a means 125 for making the thickness of a layer of plants to be treated uniform, for example for obtaining a layer five to ten centimeters thick; and
a conveyor 145 which transports the homogenized layer of ground plants to the means 155 for generating electromagnetic waves.

Optionally, the device 100 that is the subject of the invention comprises, in addition:
a means 130 for treating the foliage and stems of the plants, without utilizing the means for generating electromagnetic waves; and
a means 135 for extracting the root systems, rhizomes and contaminated earth upstream of the grinding means 120; and/or
a means 140 of cutting and transporting all of the submerged vegetative part for separate treatment on the machine.

As can be seen by reading the above description, invasive, proliferating plants can be sterilized directly where they are collected. In addition, the residues, i.e. the sterilized plants, can be left behind and used to nourish the renaturalized plants planted in their place. It is therefore unnecessary to transport these invasive plants, or to store them out of the ground.

The metals, which can interact with the electromagnetic waves generated by the generating means, are extracted upstream of this generation means. The effectiveness of the electromagnetic waves is thus increased.

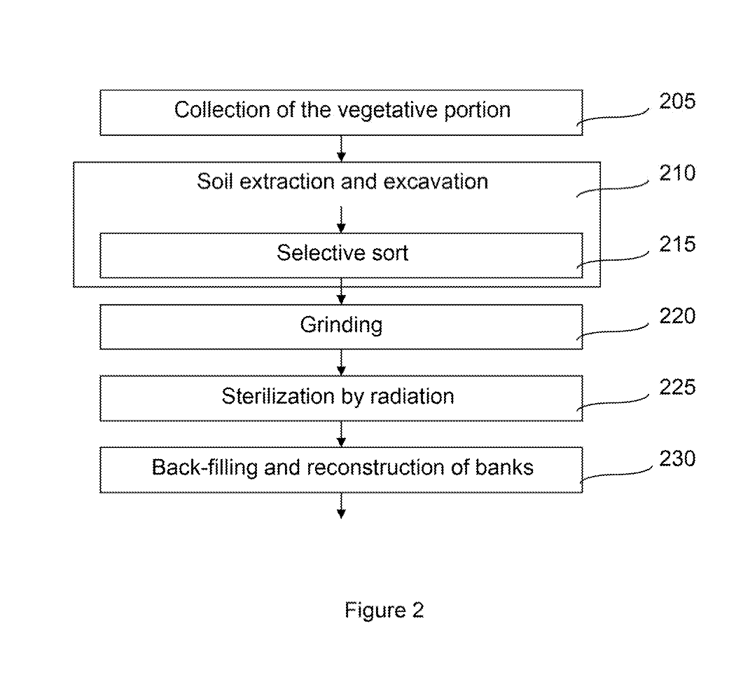

In its first embodiment, the method that is the subject of the present invention comprises first of all, as shown in FIG. 2:
a step 205 of clearing the invaded site to collect all the vegetative parts of the plants; and
a step 210 of extracting and excavating the contaminated soil.

The step 210 comprises a selective sorting step 215, for extracting the solid waste and scrap, utilizing at least one trommel and at least one electromagnet.

It is noted here that the trommel is an internally-fed sieving system. In its operating principle, the trommel is a rotating sieve with an internal feed. The effluent is brought by pumping or under the action of its weight to the inside of the strainer. For an identical flow rate, the drum is longer than a rotary sieve drum. The effluent therefore has time to spread over the entire separation surface, then pass through the strainer to reach the outlet pipe. The waste is stopped inside the cylinder and pushed in the opposite direction from the feed by a ribbon screw welded to the drum. This screening waste is then evacuated from the unit under the action of its weight. Some trommels are equipped with a rinsing ramp. Access to the strainer is facilitated by an inspection hatch equipped with a dry contact for use in complete safety. The drum consists of a wire coil with a triangular cross-section; its length varies, for example, between 1,200 and 1,800 mm and its diameter between 500 and 800 mm.

Then, a grinding step 220 is carried out, to make the residual plant mass uniform.

During a step 225, the ground-up plant mass is sterilized by heat treatment. In this way the invasive, proliferating plants are eradicated.

During a step 230, the bank is back-filled and reconstructed with the sterilized ground-up plant mass. In this way the site is renaturalized.

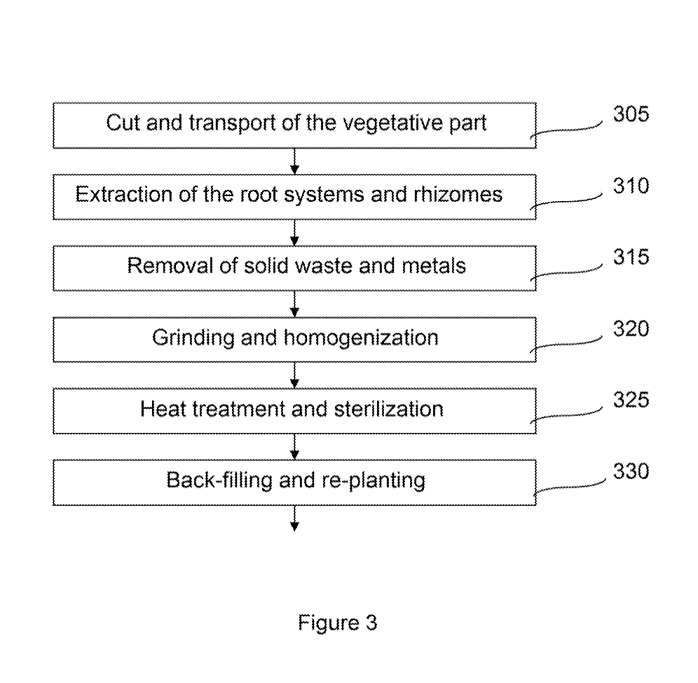

In its second embodiment, the method that is the subject of the present invention comprises first of all, as shown in FIG. 3:
a step 305 of cutting and transporting all the vegetative part, to remove the foliage and stems for separate treatment on the machine;
a step 310 of extracting the root systems, rhizomes and contaminated earth, by power shovel for extracting and loading the mobile grinder;
a step 315 of selectively sorting solid waste by trommel and electromagnet, during which the coarse material and metallic waste are discharged and removed;
a step 320 of grinding and making uniform the material into a layer five to ten centimeters thick, allowing the materials to be treated to be layered;
a step 325 of heat treatment and sterilization, then of discharging all the inert materials, ground-up inactive rhizomes; and
a step 330 of back-filling on the borrow areas, compacting the soil and re-planting using ecological engineering techniques.

Steps 305 and 310 can comprise a water-jetting step.

In this way, the treated top growth and doubtful heterogeneous gravel from a quarantine area, to which the treatment machine head returns, are kept out of the floodplain

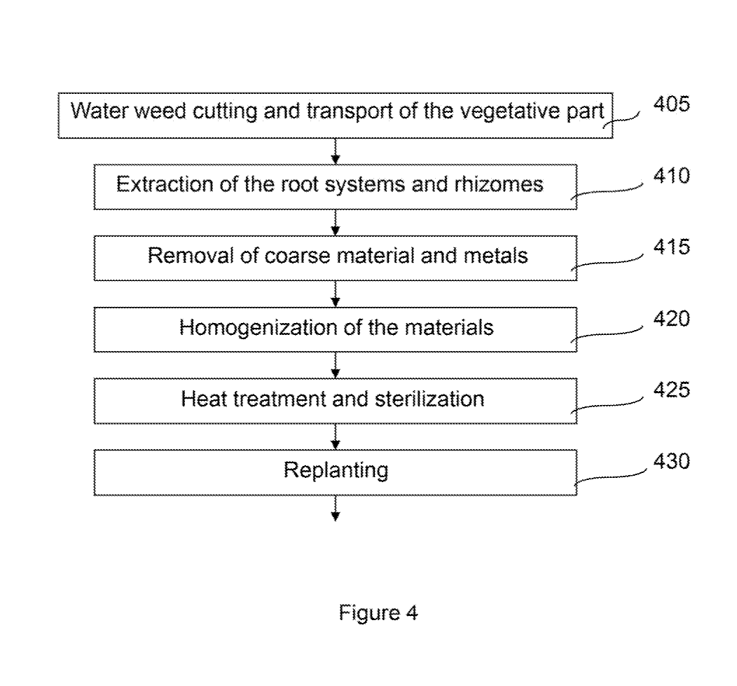

In its third embodiment, the method that is the subject of the present invention comprises first of all, as shown in FIG. 4:
a step 405 of cutting and transporting all of the submerged vegetative part during which the submerged vegetation is removed by weed cutter or pulled out manually for separate treatment on the machine;
a step 410 of extracting the root systems, rhizomes, and the contaminated sediment and silt, by under-water grinder, followed by a pump discharging the extracted materials (size less than ten centimeters);
a step 415 of selectively sorting rhizomes and wood fragments by trommel and electromagnet, during which the coarse material and metallic waste are discharged and removed;
a step 420 of making uniform the material into a layer five to ten centimeters thick, or into a feed channel of materials to be treated;
a step 425 of heat treatment and sterilization, of maintaining at temperature on output from the machine, and then of discharging all the inert materials, ground-up inactive rhizomes;
a step 430 of re-planting with native aquatic plants, using ecological engineering techniques.

Steps 405 and 410 can comprise a water-jetting step.

It is noted that the treated top growth and doubtful heterogeneous homogenates in a quarantine area are maintained in swaths.

The various steps illustrated in FIGS. 2 to 4 are detailed below.

Before the intervention of the machine for eradicating exotic plants, the area must be cleared of its foliage and stems. This is to not overload the machine with untreated plant matter and to increase its effectiveness during the subsequent treatment of the soil. During the clearing operation, every precaution must be taken not to propagate the plant on another site. The plant matter must be packaged into bundles to be engaged in the hopper of the grinder. The most suitable tool is to use a holding clamp on the arm of a power shovel.

The equipment used must be cleaned and free of any plant fragments before another use.

Exotic plants can have a deep root system (e.g. Japanese Knotweed); it is therefore necessary to dig and remove the contaminated soil to a depth of more than one meter in some areas where the plant has been present for more than 10 years. Extracting this will be done using a power shovel of more than 8 tonnes. The extracted contaminated soil must be put directly into the eradication machine.

Every precaution must be taken before the power shovel is used on another site.

With regard to the eradication operation, the machine is used for feeding the upper hopper. The contaminated soil is therefore put directly into the machine's hopper, which consists of a first sorting unit. Given the heterogeneous nature of the materials extracted, different sorting methods must be used depending on the nature and size of the materials present. In effect, the substrate can be a former disposal area or a dynamic water course alluviation, consisting of large blocks. These situations prove to be very favorable for colonization by Knotweed and are the most constraining in regard of the technical level of soil treatment, level areas with a particle size less than 10 cm being the easiest with regard to soil treatment.

Then the machine separates the scrap. An electromagnet mounted on an extraction belt positioned above the waste also allows metallic materials to be eliminated.

A trommel-type circular particle-size separator allows solid waste and large blocks, cleared of any earth or organic fragments, to be removed. This trommel, equipped with different sizes of openings, allows the materials to be sorted according to their block/particle size distribution and their weight. The largest rubble (greater than twenty centimeters, depending on the area) can be discharged directly, after being checked visually for the “absence” of any organic particles, for example agglomerated in blocks of clay. An operator therefore visually inspects this first extraction.

Most of the material is then brought on a belt to the stone crusher, which makes it possible to reduce the size of the incoming material (wood fragments, blocks less than two centimeters in diameter, clods of soil and rhizomes). This step makes it possible to homogenize the inert portion mixed with the organic matter to be treated and thus to optimize its treatment. These products are then transported on a belt in a ten centimeter thick layer to the actual treatment unit. The uniform ten centimeter thick product layer passes through a microwave oven capable of reaching a minimum temperature of 100° C. in all the molecules containing water. This quick rise in temperature causes the cell walls to burst and be destroyed. Indeed, the plant cells, and therefore the fragments of rhizomes, have a very high water content, sometimes more than 90% of the wet weight.

The soil treated in this way is sterilized of all turgescent plant cells and thus has no exotic plants. Similarly, imported topsoil and compost can be sterilized in bulk before they are re-used, and guaranteed “free of exotic plants”.

Details of the impact of the microwaves on living cells are given below. When the microwave oven is in operation, it emits polarized radiation with a frequency of between 500 GHz and 5 GHz, for example a frequency of 2.4 GHz, and preferably at a frequency of less than 1 GHz, e g. 915 MHz. The polarization vector of the wave changes at the frequency of the waves emitted. Plant cells consist mainly of water. The water molecule is what is known as a polar molecule, which means that the barycenters of the negative and positive charges are not the same. As a result, there are places that are more electronegative than others. This feature makes the water molecule sensitive to variations in the electric field produced by the microwaves. Specifically, water molecules follow the polarization of the wave, and thus change direction several billion times a second. It is this agitation of the molecules that causes the cells, which have a high water content, to heat up or even explode. The algae and turgescent root systems, such as rhizomes, are full of water, with water contents of more than 80% of the biomass.

The sterile soil and all the rhizomes, ground up and inactive, are discharged.

The method that is the subject of the present invention therefore does not require rhizomes to be stored.

With regard to the subsequent finishing actions, the treated materials are used directly as back-fill in the areas excavated previously. They are regularly compacted into a 20 to 30 centimeter thick layer, to limit as far as possible these areas being reconquered by new invasions of exotic plants. The upper soil portion put back in place is then re-planted using vegetation engineering methods, in order to give the environment back its stability and its biodiversity.

It is noted that the work prevents any risk of worsening the spread of invasive species. Any means allowing floating sprigs of primrose willow to be recovered are systematically and rigorously used (nets for mechanized operations, geotubes, dip net for manual operations, possibly straw bales, etc.).

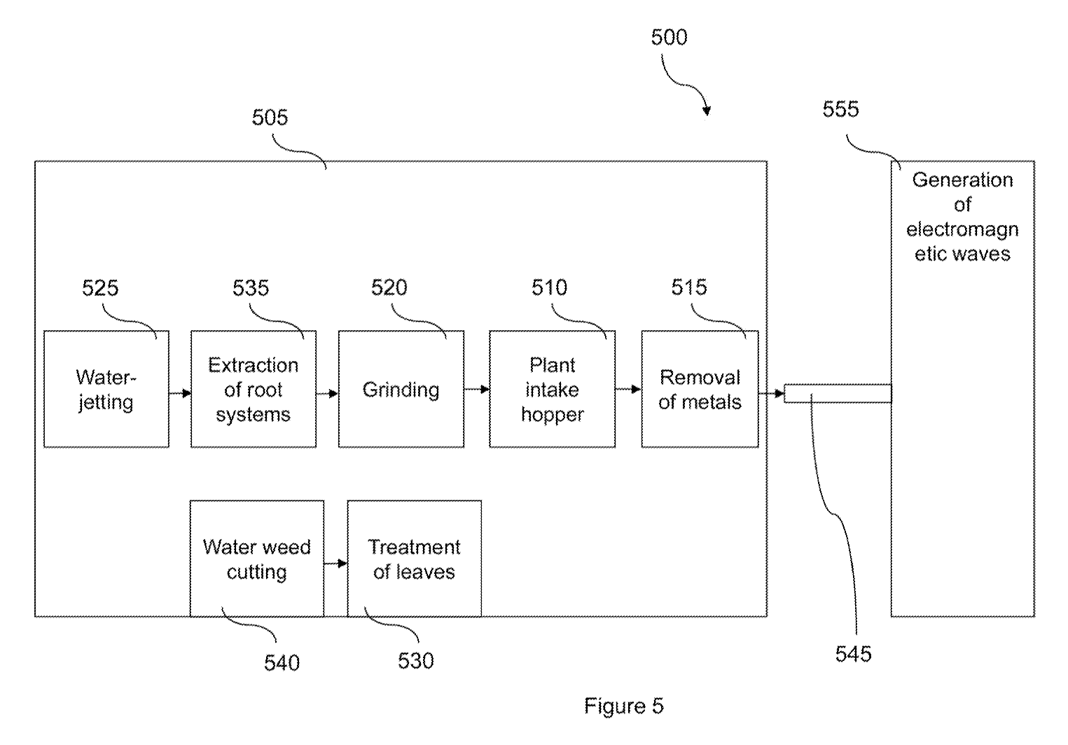

FIG. 5 shows a device 500 for eradicating plants, which comprises a means 505 for introducing the plants to be treated, and a means 555 for generating electromagnetic waves for sterilizing the plants within the layer of plants. This means 555 for generating electromagnetic waves preferably emits microwaves, and preferably with a frequency of less than 1 GHz. The means 555 for generating electromagnetic waves is designed to heat the seeds of the plants to a temperature above 60° C., and preferably above 100° C. The seeds are thus heated until they are deactivated and, preferably, all of the plant's tissues are killed.

The means 505 for introducing the plants to be treated comprises:
a means 525 for water-jetting plants, configured to locally suck up and transfer to the treatment platform all the plants and their substrate before they pass in front of the means for generating electromagnetic waves;
a means 535 for extracting the root systems, rhizomes and contaminated earth upstream of a grinding means 520. The seeds are preferably also extracted with the fine material, or separated by flotation, before heat treatment;
a means 520 for grinding the extracted portion of the plants, which makes the residual plant mass uniform; possibly, the means 520 operates by dilaceration (in water);
an accelerator pump and/or conveyor 545 which transports the ground-up plants to the treatment platform, means 555 for generating electromagnetic waves;
a means 510 for transporting plants to the ground and storing in a plant intake hopper,
a means 515 for screen raking the extracted materials and partial dehydration.

For example, the screen raking comprises a harvest screw conveyor type of conveyor, mounted on a trough, pierced over the entire underside to let materials with a dimension of less than 5 mm flow through. A trommel, as described in step 215, can also be utilized. The solid materials fall on top of the device and the fine elements, carried by the water, are taken by pumping.

Partial dehydration is obtained by geotubes or continuous band filters to trap the seeds.

All the solid materials (from the screen raking and other filters) is sent into the means 555 for generating electromagnetic waves, for example microwaves.

Optionally, the device 500 comprises, in addition:
a means 540 of cutting and transporting all of the submerged vegetative part for separate treatment on the machine; and/or
a means 530 for directly treating imported soil or compost that may contain unwanted plant fragments or seeds, WITH the utilization of the means for generating electromagnetic waves.

As can be seen by reading the above description, invasive, proliferating plants can be sterilized directly where they are collected. In addition, the residues, i.e. the sterilized plants, can be left behind and used to nourish the renaturalized plants planted in their place and selected for their use (green and landscape plantings).

In variants of the various embodiments described above, the means and step of layering the material to be treated are eliminated. In particular, using vertical tubes, for example made of Teflon (registered trademark), and smaller wave frequencies, for example less than 1 GHz, allow the matter to be treated in depth.

In variants, water-jetting (separating water from material pumped in the river or canal and its preparation by pre-washing through to the final wash, so that it can be used again) algae or plants allows them to be extracted and then partially dried before the microwave treatment. The water-jetting is a technique positioned upstream of the heat treatment method, which allows plants and silts to be pumped to a drainage station. The water-jetting, carried out using a boat, allows the plants and their seeds, the silt and the gravel to be pumped with large volumes of water.

1-15. (canceled)

16. A mobile device for eradicating at least one of proliferating and invasive terrestrial plants, comprising a feeder to introduce the plants to be treated; and a generator to generate electromagnetic waves for sterilizing the plants within a layer of plants.

17. The device according to claim 16, wherein the feeder is configured to set up the layer of plants to be treated.

18. The device according to claim 16, wherein the generator emits microwaves.

19. The device according to claim 18, wherein the generator emits microwaves with a frequency between 500 MHz and 5 GHz.

20. The device according to claim 19, wherein the generator emits microwaves with a frequency between 500 MHz and 1 GHz.

21. The device according to claim 16, wherein the generator is configured to heat seeds of the plants to a temperature above 60° C.

22. The device according to claim 16, further comprising a hopper to receive the plants; and a separator to remove at least one of metal and stones.

23. The device according to claim 16, wherein the feeder is configured to make the a thickness of the layer of plants to be treated uniform, which is arranged upstream from the generator.

24. The device according to claim 16, further comprising a grinder to grind the plants, which is arranged upstream from the generator.

25. The device according to claim 16, wherein the feeder is configured to water-jet the plants and partially dry the plants before they pass in front of the generator.

26. A method for eradicating at least one of proliferating and invasive terrestrial plants, comprising the steps of:
collecting the plants;
introducing the plants to be treated; and
generating electromagnetic waves for sterilizing the plants within a layer of plants.

27. The method according to claim 26, further comprising the step of setting up the layer of plants to be treated.

28. The method according to claim 26, further comprising the step of generating microwaves.

29. The method according to claim 28, further comprising the step of generating microwaves with a frequency between 500 MHz and 5 GHz.

30. The method according to claim 29, further comprising the step of generating microwaves with a frequency between 500 MHz and 1 GHz.
